(19) United States
(12) Patent Application Publication
(10) Pub. No.: US 2026/0007343 A1
(43) Pub. Date: Jan. 8, 2026

(54) MOUTHGUARD FOR ELECTROOCULOGRAM SIGNAL MEASUREMENT

(71) Applicant: THE ROYAL INSTITUTION FOR THE ADVANCEMENT OF LEARNING/MCGILL UNIVERSITY, Montréal (CA)

(72) Inventors: Sharmistha BHADRA, Montreal (CA); Han Cat NGUYEN, Montreal (CA)

(21) Appl. No.: 19/261,344

(22) Filed: Jul. 7, 2025

Related U.S. Application Data
(60) Provisional application No. 63/668,419, filed on Jul. 8, 2024.

(51) Int. Cl. A61B 5/16; A61B 5/00

(52) U.S. Cl. CPC A61B 5/163; A61B 5/682; A61B 2560/0214

(57) ABSTRACT

A mouthguard for measuring eye movements of a wearer, has: a body adapted to engage teeth of the wearer and extending laterally along a frontal axis between a left side and a right side and extending vertically along a vertical axis, between a top and a bottom; a power source; electrooculogram (EOG) electrodes secured to the body and operatively connected to the power source, the EOG electrodes including a top right electrode, a top left electrode, at least one bottom electrode, and a bias electrode being substantially centered on the body; and a controller operatively connected to the EOG electrodes and to the power source, the controller having a processing unit and a computer-readable medium having instructions stored thereon executable by the processing unit to: receive electrooculogram signals from the EOG electrodes; and analyse the electrooculogram signals to determine movements of one or more eye of the wearer.

### CROSS-REFERENCE TO RELATED APPLICATIONS

The present application claims priority on U.S. Patent Application No. 63/668,419 filed on Jul. 8, 2024, the entire content of which is incorporated by reference herein.

### TECHNICAL FIELD

The present application relates generally to systems and methods for sleep monitoring.

### BACKGROUND

Sleep quality and disorders have been assessed and diagnosed based on sleep-stages, which may be classification using on a combination of polysomnography (PSG), electroencephalography (EEG), electrooculography (EOG), and electromyography (EMG). Currently, there are sleep monitoring wearables in the forms of headbands, sleep masks, and in-ear devices, which are used for such sleep-stage classification. However, many of these wearable devices can be uncomfortable and tend to get displaced during sleep, which results in less accurate or incorrect measurements. Moreover, commercially available devices are still expensive. Hence, although existing solutions may be satisfactory to some extent, there remains a need for improvement.

### SUMMARY

Monitoring electrooculogram (EOG) signal alone is sufficient for accurate sleep-stage classification (accuracy of more than 80%) since the eyes move differently in different sleep stages. A sleep-monitoring EOG system may be simpler in design, more compact, faster to set up, and more comfortable for a user to wear. It may need less data storage space and may use lighter machine learning models for sleep-stage classification than PSG data. Based on the patterns of the sleep stages, many sleep disorders such as REM sleep behavior disorder (RBD), sleep apnea, and narcolepsy and diseases such as Alzheimer's and epilepsy may be diagnosed early.

In this disclosure, a smart mouthguard that can monitor intra-oral EOG signals is disclosed. The device acquires the EOG signal from the inner side and surroundings of the upper and lower lip areas. In one particular embodiment, the proposed smart mouthguard comprises of two sub-mouthguards, although an embodiment using a single sub-mouthguard is also disclosed, five soft conductive fabric electrodes, and an EOG measurement board implemented on a flexible substrate. The measurement system is battery-operated and sends EOG data wirelessly. The quality of the EOG signal acquired by the smart mouthguard is good enough to clearly determine and differentiate the processed intra-oral EOG signal patterns corresponding to different eye activities. With its comfort, low cost, and reliable wireless transmission, the EOG mouthguard has great potential for EOG-based sleep monitoring.

In one aspect, there is provided a mouthguard for measuring eye movements of a wearer, comprising: a body adapted to engage teeth of the wearer, the body extending laterally along a frontal axis between a left side and a right side and extending vertically along a vertical axis normal to the frontal axis, between a top and a bottom; a power source; electrooculogram (EOG) electrodes secured to the body and operatively connected to the power source, the EOG electrodes including a top right electrode on the right side and adjacent the top of the body, a top left electrode on the left side and adjacent the top, at least one bottom electrode mounted adjacent the bottom of the body, and a bias electrode being substantially centered on the body between the left side and the right side; and a controller operatively connected to the EOG electrodes and to the power source, the controller having a processing unit and a computer-readable medium operatively connected to the processing unit and having instructions stored thereon executable by the processing unit to: receive electrooculogram signals from the EOG electrodes; and analyse the electrooculogram signals to determine movements of one or more eye of the wearer.

In certain embodiments, the mouthguard for measuring eye movements of a wearer as defined above and described herein also includes one or more of the following features, in whole or in part, and in any combination.

In some embodiments, the at least one bottom electrode includes: a bottom right electrode on the right side and adjacent the bottom of the body; and a bottom left electrode on the left side and adjacent the bottom of the body.

In some embodiments, the computer-readable medium has instructions executable by the processing unit to determine a sleep stage of the wearer based on the movements of the one or more eye determined by the electrooculogram signals.

In some embodiments, the computer-readable medium has instructions executable by the processing unit to determine the sleep stage by determining that the sleep stage corresponds to: wake stage, stage 1 (n1), stage 2 (n2), stage 3 (n3), and rapid eye movement (rem) stage.

In some embodiments, the EOG electrodes include conductive fabric electrodes.

In some embodiments, the computer-readable medium has instructions executable by the processing unit to receive the electrooculogram signals from the EOG electrodes via four channels.

In some embodiments, the computer-readable medium has instructions executable by the processing unit to analyse the electrooculogram signals by performing a fourth-order wavelet decomposition for each of the four channels.

In some embodiments, the computer-readable medium further instructions executable by the processing unit to: receive a bias signal from the bias electrode; and subtract the bias signal from the electrooculogram signals.

In some embodiments, the mouthguard for measuring eye movements of a wearer includes a wireless communication module for data transmission.

In some embodiments, the mouthguard for measuring eye movements of a wearer further includes an inertial measurement unit (IMU) sensor mounted to the body, the IMU sensor operatively connected to the controller, the computer-readable medium further having instructions executable by the processing unit to: receive IMU signals from the IMU sensor; and remove artifacts from the electrooculogram signals using the IMU signals, the artifacts caused by one or more of jaw clenching, snoring, tongue movement, gulping, and head movements of the wearer.

In some embodiments, the power source is a battery mounted to the body.

In some embodiments, the mouthguard for measuring eye movements of a wearer includes an inductive charging coil mounted to the body and operatively connected to the battery, the inductive charging coil configured to wirelessly recharge the battery.

In some embodiments, the body is configured to be engaged solely by either of upper teeth and lower teeth of the wearer.

In some embodiments, the body has an upper part and a lower part, the upper part configured to engage upper teeth of the wearer, the lower part configured to engage lower teeth of the wearer.

In another aspect, there is provided a method for analysing sleep, the method comprising: receiving electrooculogram signals from EOG electrodes within a mouthguard worn by a wearer; analysing the electrooculogram signals to determine movements of one or more eye of the wearer; and determining a sleep stage of the wearer based on the movements of the one or more eye.

In certain embodiments, the method for analysing sleep as defined above and described herein also includes one or more of the following features, in whole or in part, and in any combination.

In some embodiments, the mouthguard has a left side, a right side, a top, and a bottom, the EOG electrodes including a top right electrode on the top and right side, a top left electrode on the top and left side, a bottom right electrode on the bottom and right side, and a bottom left electrode on the bottom and left side, said analysing of the electrooculogram signals to determine the movements of the one or more eye includes: determine left and right movements of the one or more eye from potential differences between the EOG electrodes located on the top and/or between the EOG electrodes located on the bottom of the mouthguard; and determine up and down movements of the one or more eye from potential differences between the EOG electrodes located on the left side and/or between the EOG electrodes located on the right side of the mouthguard.

In some embodiments, the determining of the sleep stage of the wearer based on the movements of the one or more eyes includes determine that the sleep stage corresponds to one or more of: wake stage, stage 1 (n1), stage 2 (n2), stage 3 (n3), and rapid eye movement (rem) stage.

In some embodiments, the analysing of the electrooculogram signals includes: performing one or more of a wavelet decomposition, a butterworth bandpass filtering, and a multivariate empirical mode decomposition, for each of four channels, each of the four channels associated with a respective one of the EOG electrodes.

In some embodiments, the EOG electrodes further include a bias electrode, the method comprising: receive a bias signal from the bias electrode; and subtract the bias signal from the electrooculogram signals.

In some embodiments, the mouthguard further includes an inertial measurement unit (IMU) sensor, the method comprising: receive IMU signals from the IMU sensor; and remove artifacts from the electrooculogram signals using the IMU signals, the artifacts caused by one or more of jaw clenching, snoring, tongue movement, gulping, and head movements of the wearer.

### BRIEF DESCRIPTION OF THE DRAWINGS

Reference is now made to the accompanying figures in which:

FIG. 1A is a cross-sectional view of a human eye;

FIG. 1B is a front view of a portion of a face of a user;

FIG. 2 is a schematic representation of a mouthguard for sleep analysis;

FIGS. 2A to 2C are three dimensional views of a mouthguard for sleep analysis;

FIG. 2D is a front view of a data acquisition module of the mouthguard of FIG. 2A;

FIG. 2E is a rear view of the data acquisition module of FIG. 2D;

FIG. 2F is a three-dimensional view of the mouthguard of FIG. 2A on a holder for recharging;

FIG. 3 is a schematic view illustrating the different components of a system for sleep-stage monitoring including the data acquisition module of FIG. 2D;

FIG. 4A are graphs illustrating a wavelet decomposition of the electrooculogram measured using the mouthguard of FIG. 2A;

FIG. 4B are graphs illustrating another wavelet decomposition of the electrooculogram measured using the mouthguard of FIG. 2A;

FIG. 5A are graphs illustrating processed intra-oral electrooculogram data of five eye movement activities obtained from a horizontal channel of the data acquisition module of FIG. 2D;

FIG. 5B are graphs illustrating processed intra-oral electrooculogram data of five eye movement activities obtained from a vertical channel of the data acquisition module of FIG. 2D;

FIG. 6A is a front view of a face of a user illustrating electrode placement for testing purposes;

FIG. 6B is a graph illustrating variations of impedance as a function of frequency for two types of sensors located either in the mouth of the user or on a forehead of the user;

FIGS. 7A to 7E illustrate manufacturing steps of one of the electrodes of the mouthguard of FIG. 2A;

FIG. 8A is a three-dimensional view of a mouthguard in accordance with another embodiment;

FIG. 8B is a front view of a measuring board of the mouthguard of FIG. 8A;

FIG. 8C is a back view of the board of FIG. 8B;

FIG. 9 are graphs illustrating electrooculogram patterns for different channels and for different movements performed by eyes of a wearer of the mouthguard of FIG. 8A;

FIG. 10 are graphs illustrating electrooculogram patterns illustrating artifacts caused by movements of the wearer of the mouthguard of FIG. 9A;

FIG. 11 are graphs illustrating electrooculogram patterns for each of the channels before and after artifact removal; and

FIG. 12 is a flowchart illustrating steps of a method of analysing sleep of a wearer of the mouthguard;

FIG. 13 is a schematic representation of a controller of the mouthguard.

### DETAILED DESCRIPTION

### Introduction

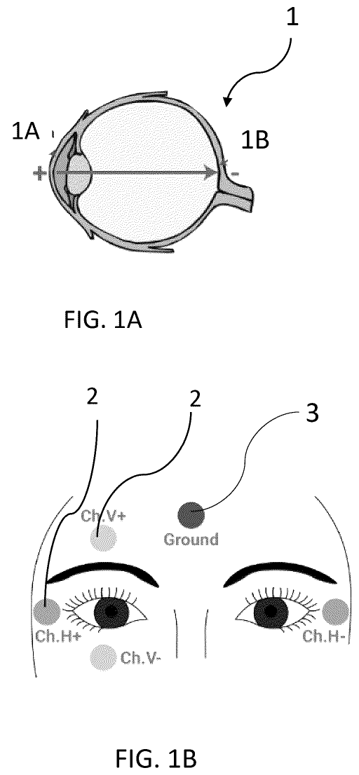

Referring to FIGS. 1A and 1B, an eye is shown at 1. Electrooculogram (EOG) measures the cornea-retinal potential (CRP) between the eye cornea 1A and the ocular fundus 1B (including retina) of the eye 1. Due to the electrical activities of the photoreceptors and neurons in the retina, the ocular fundus 1B is more negative in potential than the cornea 1A. Thus, CRP can be considered as a dipole. When the eye 1 moves, the CRP changes, which causes changes in the EOG signal captured by electrodes 2 disposed around the eye 1. When the eye 1 moves, the CRP dipole rotates. If the electrodes 2 are placed around the eyes 1, the electrode 2 that becomes closer to the cornea 1A will have its electrical potential increased, while the electrode that becomes nearer to the retina 1B will have its electrical potential decreased. Thus, the potential difference between the two electrodes will change, which leads to hyperpolarization or depolarization in the EOG signal. The amplitude of the EOG signal becomes smaller when the electrodes 2 are placed farther from the eyes. Additionally, the EOG signal amplitude may almost be linearly correlated to the angle the eye rotates, especially within +/−35 degrees horizontally and +/−45 degrees vertically.

As shown in FIG. 1B, the EOG channel measuring the potential difference between the two electrodes above and below the eyes captures the vertical eye movements, while the channel measuring the potential difference between the two electrodes on the left and right sides of the eyes detects the horizontal eye movements. Their combination may allow monitoring eye movement in all directions. A ground or bias electrode 3 may be positioned on a middle line of the forehead for reference or common noise reduction.

Thus, it may be possible to detect movements of the eyes to perform sleep-stage classification, which can be used for sleep quality assessment and early diagnoses of many diseases such as Alzheimer's, epilepsy, and sleep apnea, amongst others. Since the eyes move differently during different sleep stages, EOG data alone is sufficient for accurate sleep-stage classification, and thus, for sleep monitoring.

According to the guidelines of the American Academy of Sleep Medicine (AASM), a sleep cycle occurs in five stages: wake, stage 1 (N1), stage 2 (N2), stage 3 (N3), and Rapid Eye Movement (REM) stage. Traditionally, sleep scoring (identifying when each sleep stage happens) is done by analyzing polysomnographic (PSG) data which includes EEG, EOG, EMG, ECG, and pulse oximetry (SpO2). However, to obtain all of those biopotential data, many systems and electrodes (usually with long wires) need to be placed on the patient, which leads to time-consuming set up and may decrease the sleep quality of the wearer. Moreover, EEG electrodes placed on the side and back of the head tend to cause discomfort to the user when they lie down on the electrodes during sleep. Moreover, EOG data alone may be sufficient

These days, affordable intra-oral wearables such as mouth-guards and mandibular advanced devices are already worn by many people during sleep to reduce bruxism (teeth grinding), snoring, and mild sleep apnea. Therefore, an EOG-measuring intra-oral device may offer sleep monitoring at a low cost without causing discomfort to the user. The mouthguard may include one or two sub-mouthguards, five soft conductive fabric electrodes, and a flexible EOG measuring board. The EOG-measuring intra-oral device may include only one mouthguard as will be discussed below. It may be battery-operated and uses a microprocessor-Bluetooth module for wireless EOG data transmission. The device may be charged wirelessly using inductive charging. It has been validated for different eye movements. The proposed EOG monitoring platform can be integrated in diverse intra-oral wearables and has the potential for comfortable sleep monitoring. The present disclosure presents a mouthguard that may be used for sleep analysis and that may at least partially alleviate these drawbacks.

### Mouthguard

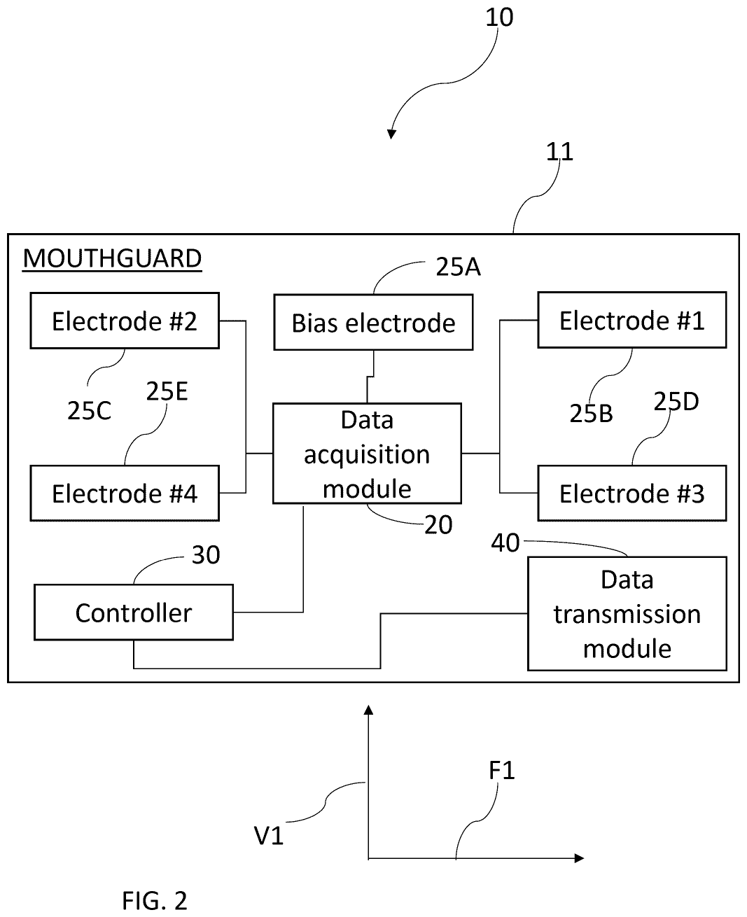

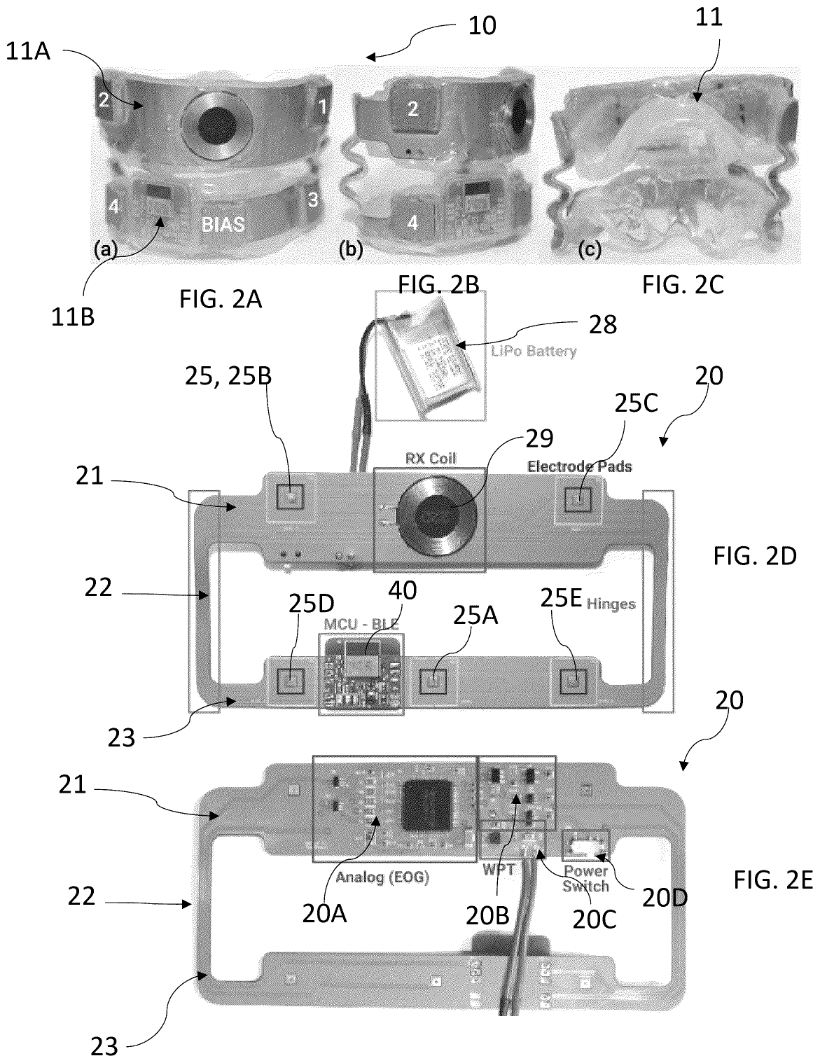

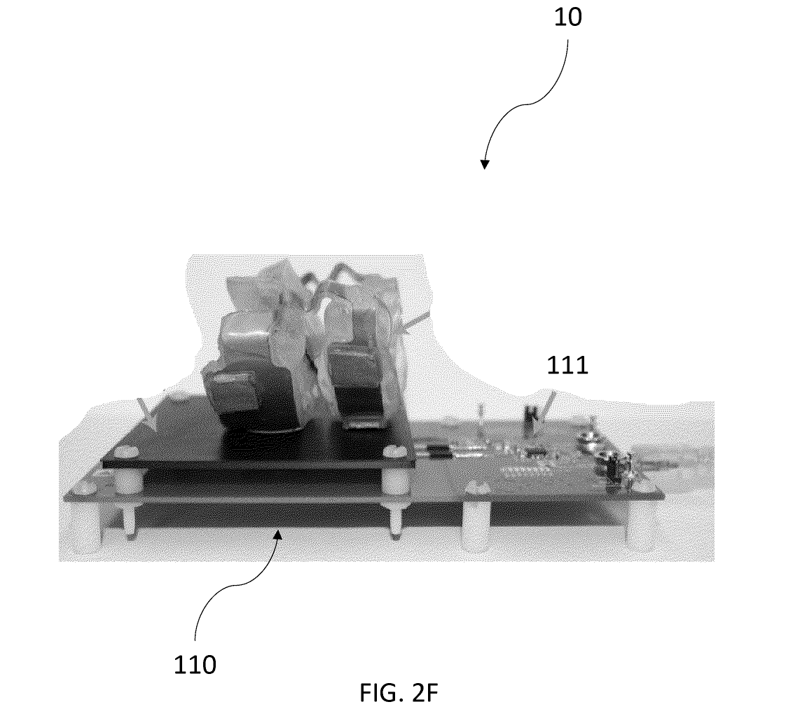

Referring to FIG. 2, a schematic representation of a mouthguard is shown at 10. The mouthguard 10 is configured to be worn by a user while sleeping. The term “mouthguard” as used herein is understood to mean any intra-oral wearable device, whether this device takes the form of traditional mouthguard (which may cover and protect the upper and/or lower teeth) or another type of intra-oral device that can be worn by the user during sleep and which is located proximate to the upper and lower lips of the wearer. The mouthguard 10 includes a body 11, which has a shape configured to be engaged by one or more of upper and lower teeth of a wearer. The body 11 may have two parts, such as a lower part and an upper part, to engage the lower teeth and the upper teeth, respectively. The body 11 further acts as a substrate onto which are affixed other components of the mouthguard 10. The body 11 extends along a vertical axis V1, which may be perpendicular to a ground when the wearer is standing, and a frontal axis F1, which is perpendicular to the vertical axis V1 and that extends between left and right sides of the wearer. The mouthguard 10 is equipped with electrodes, namely a bias electrode 25A, an upper left electrode 25B, an upper right electrode 25C, a lower left electrode 25D, and a lower right electrode 25E.

The mouthguard 10 includes a data acquisition module 20 configured for detecting the movements of the eyes of a user to perform sleep analysis. The electrodes are operatively connected to the data acquisition module 20. The mouthguard 10 further includes a data transmission module 40, also referred to as a communication module, operatively connected to the controller 30 and configured for transmitting the data acquired by the data acquisition module 20 and the electrodes.

Referring to FIGS. 2A-2E, the mouthguard 10 is shown in greater details. In the embodiment shown, the body 11 may be made of flexible ethylene vinyl acetate (EVA). As shown in FIG. 2C, the body 11 may have a shape suitable for mating with teeth of the user. The body 11 may include an upper part 11A configured to engage the upper teeth of a wearer and a lower part 11B configured to engage lower teeth of the wearer. The body 11 may be thermally molded to the top and bottom teeth and gums of the user. As will be described below, the body 11 may include solely the upper part 11A or solely the lower part 11B.

In the embodiment shown, the data acquisition module 20 is assembled from three printed circuit board (PCB) parts, namely, a top part 21, which may include 4-layer of Polyimide flex, a connector part 22, which may include 2-layer of Polyimide flex, and a bottom part 23, which may include 2-layer of FR4 TG150 rigid. The top part 21 may contain an analog front-end 20A and a power supply circuit. The bottom part 23 may house the communication module 40, which may be a microprocessor-Bluetooth module. The connector part 22 is used to connect the top art 21 to the bottom part 23. The connector part 22 may be bendable to allow movements of a mouth of the user. In other words, the connector part 22 acts as a hinge to permit the user to open and close his or her mouth. The EOG electrodes 25 (or simply “electrodes” 25) are secured to both of the top part 21 the bottom part 23. It will be appreciated that the data acquisition module 20 may include solely one of the top and bottom parts.

The bias electrode 25A may be located on the bottom part 23 and laterally substantially centered on the body between the left and right side. Herein, the expression “substantially” implies light deviations caused by, for instance, manufacturing tolerances. The bias electrode 25A may alternatively be located on the top part 21. In some embodiments, more than one electrode may be located at each of these locations (i.e., upper right, upper left, etc.). The electrodes 25 may be silver-silver chloride (Ag/AgCL) or gold (Au) electrodes used with a conductive gel in EOG measurement to ensure good skin-electrode contact with impedance below 5 kΩ (between 20 and 40 Hz). The electrodes may be embedded within a material of the body 11.

Although the mouthguard 10 includes 5 electrodes total, two upper electrodes 25B and 25C, two lower electrodes 25D and 25E, and the bias electrode 25A, the mouthguard 10 may include fewer electrodes. For example, in a particular embodiment, the mouthguard 10 may include the upper left electrode 25B, the upper right electrode 25C, the bias electrode 25A located between the upper left and the upper right electrodes, and only one bottom electrode, which may for example be centrally located. In another embodiment, the bias electrode may be located on the bottom part of the body 11, or may alternately be substituted by another suitable means configured for capturing noise generated by an environment around the wearer, whether located directly on the mouthguard or elsewhere in the system.

In an embodiment, the data acquisition module 20 is comprised of three main parts: the analog front-end 20A, the microprocessor (MCU)-BLE unit (e.g., communication module 40), and the power supply unit which includes a voltage regulator 20B, wireless power transfer (WPT) unit 20C, a Rx coil 29, and a battery 28 as shown in FIG. 2D. The battery 28 is operatively connected to the different components of the data acquisition module 20 to provide power thereto. The battery 28 may be a LiPo battery and supplies power to the board at about 3.7 V. Other voltage values are contemplated. The battery 28 may be charged wirelessly through resonant inductive coupling. A coil (e.g., Rx coil) 29 is used for this purpose. The coil 29 may be disposed on the top part 21 of the data acquisition module 20, but may alternatively be located on the bottom part 23.

The analog front-end 20A and the power supply unit 20B, 20C are both positioned on the top part 21 of the 4-layer polymide flexible PCB. They may alternatively be located on the bottom part 23. The communication module 24 is placed on the rigid 2-layer FR4 PCB. The two flexible-PCB thin hinges connects the top and bottom sub-mouthguards together while ensuring that the user can still open and close their mouth while wearing the mouthguard. A switch 20D may be provided on the top art 21 or the bottom part 23 and used to power on or off the mouthguard 10.

Referring to FIG. 2F, a holder 110 is used to wirelessly recharge the battery 28 through resonant inductive coupling. On the receiver side, the battery 28 is connected to a chip (e.g., LTC4124 chip) which connects to a 12.6 pH Rx coil (e.g., 760308101220) and a 47 nF capacitor in parallel. The operating resonant frequency is about 1.3 MHZ. The charging current and the charging voltage may be set to 100 mA and 4 V, respectively. The holder 110 includes a transmitter 111 (e.g., DC2773A-B board as part of the DC2770A-B-KIT which uses the LTC4125 chip and a 6.8 μH Tx coil (760308101104)). Other suitable transmitters may be used. During charging, the mouthguard 10 is placed on top of the holder 110 with an alignment hole. With this setup, the Rx coil on the mouthguard 10 and the Tx coil on the transmitter board 111 may be aligned concentrically and at a distance of 5 mm.

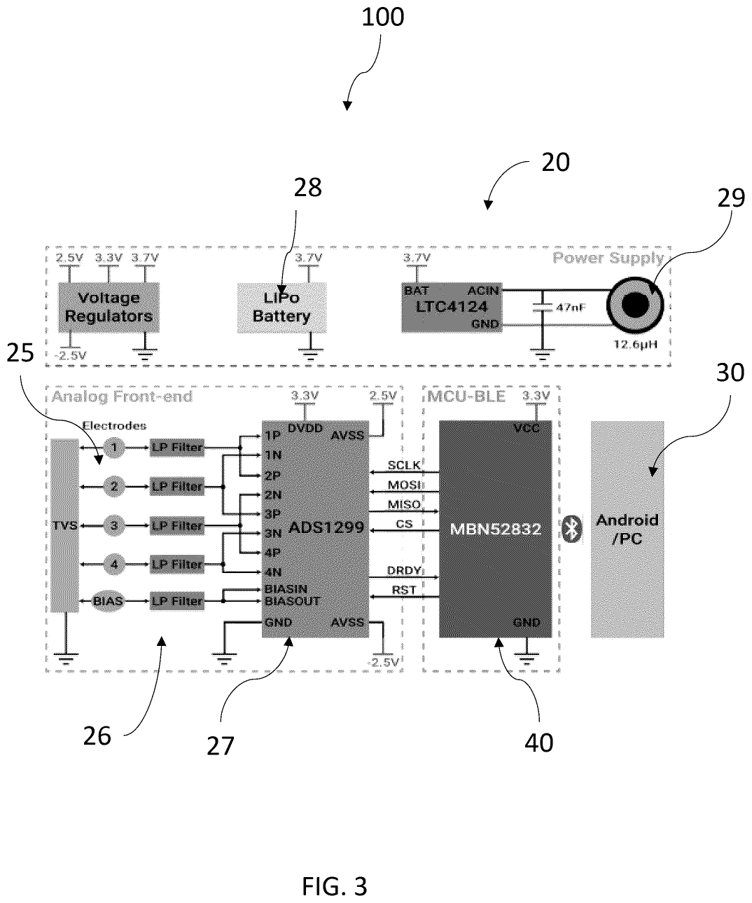

Referring now to FIG. 3, a block diagram of a system for sleep-stage monitoring is shown at 100. The electrodes 25 on the mouthguard acquire the intraoral EOG signals. Filters 26, which may be low-pass filters, may be operatively connected to the electrodes 25 to low-pass filter the signals at about 72 kHz. The filters 26 may be first-order RC filters (e.g., resistor of 2.2 KΩ and capacitor of 1 nF) to remove high-frequency noises and to safeguard the downstream electronic components from overcurrent. Then, an amplifier 27 is operatively connected to the filters 26. The amplifier 27 may be a ADS1299-4PAG used for amplification (e.g., gain of 24×(223−1)), high-pass filtering (e.g., by DC coupling), low-pass Delta-Sigma decimation filter (e.g., at 125 Hz to avoid aliasing), common-mode rejection (e.g., by driven-right-leg circuit), and digitization.

Then, the amplifier 27 is operatively connected to the communication module 40, which receives the digital data. The communication module 24 may be a Bluetooth™ module (e.g., MBN52832 5.0 module with built-in ARM Cortex M4 core), which may be a low-energy module. SPI communication at the sampling rate (sampling frequency) of 250 Hz may be used for this purpose. In some embodiments, the controller 30 may establish a Bluetooth™ pairing with the communication module 40. The controller 30 may be used to record the data. The recording may be started or stopped with terminal commands. Suitable regulators, inverters, and amplifiers may be used to interconnect the battery 28 to the different components.

After being programmed and encapsulated with bio-compatible polymer, the data acquisition module 20 and its components are attached to the body 11 such that the top and bottom parts 21, 23 are aligned with the upper part 11A and the lower part 11B of the body 11 of the mouthguard 10. In this configuration, the electrodes 25 acquire the EOG signals from the inner side and surroundings of the upper and lower lip areas. The mouthguard 10 may have four EOG channels in which two of them are for horizontal eye movement detection and two of them are for vertical eye movement detection. These channels may be bipolar channels. The BIAS electrode 25A is used for reducing common noises on all channels (such as the AC 60 Hz noise) through driven-right-leg circuit in the amplifier 27.

### Data Acquisition

EOG signals are small in amplitude, usually in the range of 50-3500 μV. Thus, EOG signals are very susceptible to noises from both the system itself and the surrounding environment. Their useful frequency components are typically in the range of 0 Hz to 38 Hz, which overlap with that of electroencephalogram (EEG) from neural activities, electromyogram (EMG) from muscle activities, and electrocardiogram (ECG) from heart activities. Therefore, besides the common surrounding noises such as the power line interference (50 Hz or 60 Hz), EOG signals are also affected by the other biopotential artifacts (EEG, EMG, and ECG). Additionally, when the user moves which causes electrode shifting or when the user sweats which changes the dielectric between the electrode and the skin, the skin-electrode impedance changes. This creates noises, especially the baseline drift, in the signals.

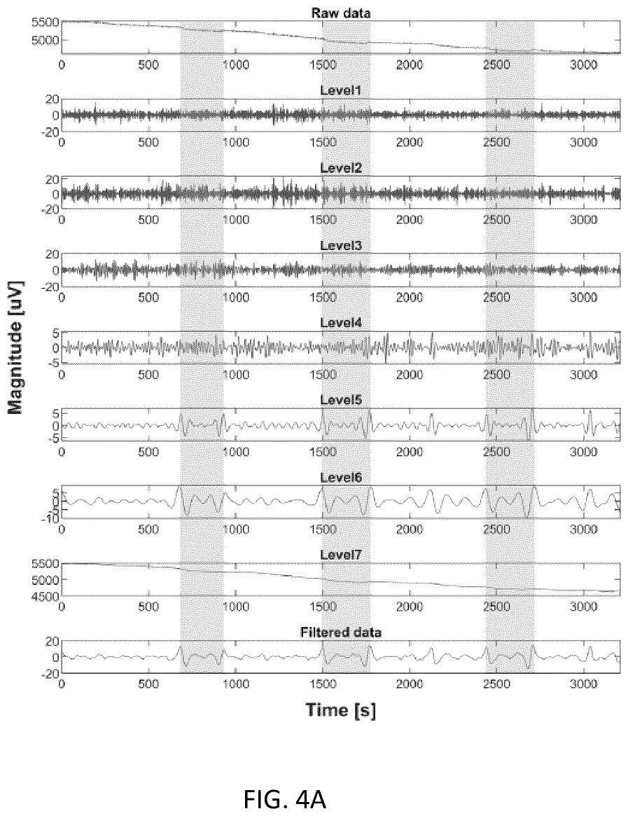

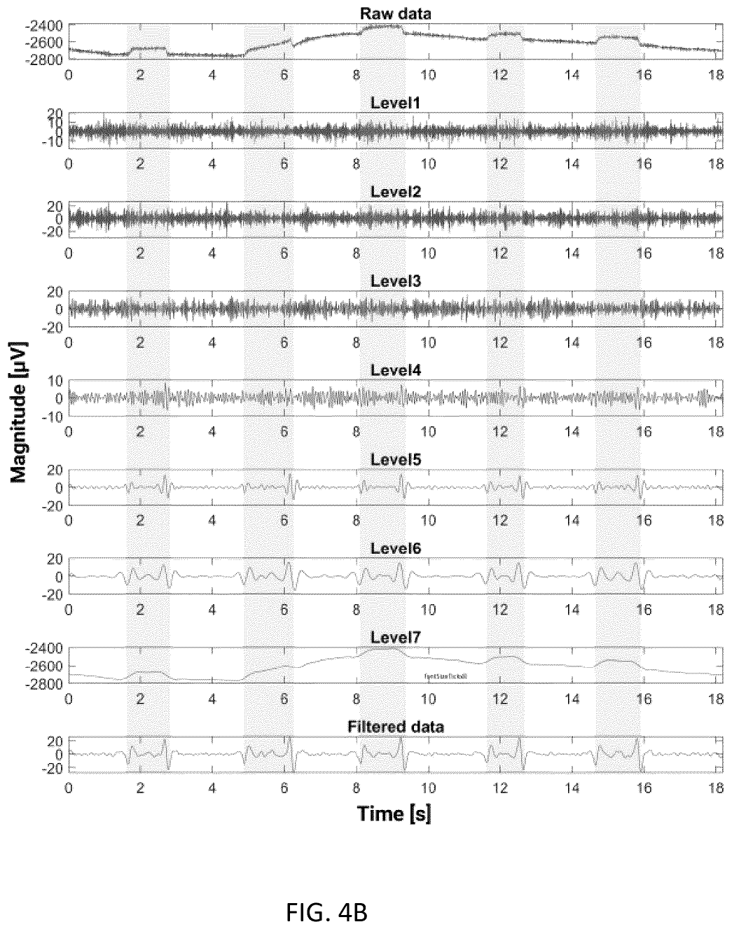

The controller 30 is used to process the data received from the electrodes 25. The fourth-order symlet wavelet decomposition was applied to remove the baseline drift (DC noise) and high-frequency noises in the acquired intra-oral EOG data. Each of the four channel signals was decomposed into seven level components (1 to 7): 62.5 Hz to 125 Hz, 31.25 Hz to 62.5 Hz, 15.625 Hz to 31.25 Hz, 7.8125 Hz to 15.625 Hz, 3.90625 Hz to 7.8125 Hz, 1.953125 Hz to 3.90625 Hz, and 0 Hz to 1.953125 Hz. The final (filtered) signal was the combination of the fifth- and sixth-level components since it was found to be the best to reduce the noises while preserving the EOG pattern features. As shown in FIG. 4A, the baseline drift and the high-frequency noises could be observed in level 7 and levels 1 to 4 respectively. Due to the discrete and periodic nature of the discrete wavelet transform, there were periodic signals added to the event features in the filtered signal. Nevertheless, the processed EOG patterns were consistent between same-eye-activity events and distinguishable between different eye movements.

As shown in FIG. 4B, a wavelet decomposition of an EOG recording from channel 1 in which the subject has looked to the left 5 times is illustrated. The high-frequency noises and the baseline drift are observable in level 1 to 4 components and level 7 component, respectively. Thus, the processed EOG signal is the combination of level 5 and level 6 components since they preserve the EOG patterns while having almost no noise left.

### Results and Discussion

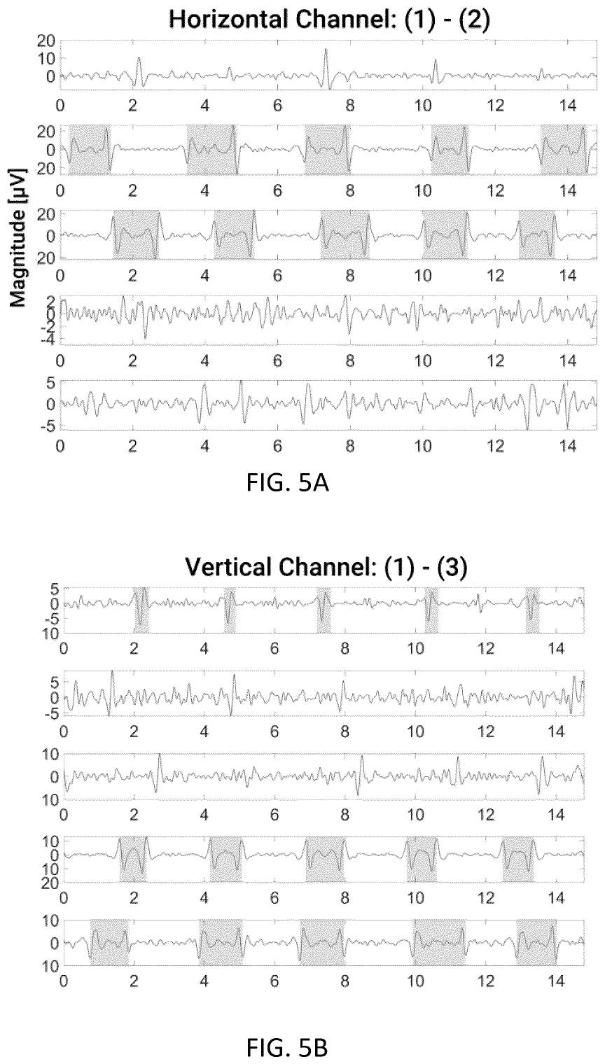

The EOG signals captured by the mouthguard 10 for five different eye activities are shown in FIGS. 5A and 5B. These eye activities are, from top to bottom, five blinks, five looks to the left, five looks to the right, five looks upward and five looks downward. The intra-oral EOG signals were smaller in magnitude comparing to the EOG signals in traditional configuration. Nonetheless, the EOG patterns corresponding to the horizontal eye movements (FIG. 5A) such as looking to the left or right were discernible in the horizontal channel (potential difference between the upper electrodes 25B, 25C or the lower electrodes 25D, 25E and the EOG patterns due to vertical eye movements (FIG. 5B) such as looking up or down and blinking could be observed in the vertical channel (potential difference between the left electrodes 25B, 25D or the right electrodes 25C, 25E). The blinking pattern is represented as a drop in voltage followed by a small peak in the vertical channels. These features have been observed for all participants.

It was observed that the horizontal eye movement patterns can be observed clearly in all trials (accuracy of 100%). However, the vertical eye activities have lower accuracy of detection from the EOG signal than the horizontal eye activities, especially the looking down eye activity with an accuracy of 94%. The vertical eye movement patterns tend to have smaller amplitude than the horizontal eye activities. This is likely due to the smaller distance between the two electrodes measuring vertical eye activities than the two electrodes measuring horizontal eye activities.

Although the intraoral electrode setup with the smart mouthguard is less affected by the movement artifacts than the standard placement on the forehead or around the eyes, the intraoral EOG signals still suffer from signal distortion due to some motion artifacts. During sleep, artifacts due to jaw clenching, snoring, tongue movement, gulping, and head movements can occur. Other signal processing methods such as multivariate empirical mode decomposition (MEMD), canonical correlation analysis (CCA), and artifact subspace reconstruction (ASR) may be used and developed to remove motion artifacts from the intraoral EOG signals.

The ocular dipole theory supported the observed EOG patterns in FIGS. 5A-5B. The electrode that got nearer to the eye cornea during the eye movement became more positive in potential. For example, in the looking-left activity, the cornea rotated to the left. Since the upper left electrode 25B was nearer to the cornea than the upper right electrode 25C, the potential at the upper left electrode 25B became more positive than that at the upper right electrode. Thus, the potential difference of the upper left electrode 25B subtracting the upper left electrode 25C became more positive, hence, a peak in the signal. On the other hand, in the looking-up activity, the ocular fundus (more negative than cornea) rotated to the bottom which was nearer to the electrodes. Since the upper left electrode 25B was nearer to the eye, it became more negative in potential than the lower left electrode 25D. Therefore, the potential difference of these electrodes became more negative, which resulted in a drop in the signal.

According to the participant, the mouthguard 10 was comfortable to wear during the experiments. The data could be transmitted wirelessly reliably and at low power consumption of only 44.5 mW. A wireless power transfer (inductive coupling) may be integrated into the system for charging the battery 28.

### Validation

Eight subjects with age between 20 and 45 and body mass index between 19 and 28 have been recruited for this study. Experimental procedures adhere to the Declaration of Helsinki. A skin-electrode impedance of the fabric EOG electrodes on the mouthguard 10 have been measured by applying a 1 mV ac current (20 to 100 Hz) through two electrodes attached to the mouthguard 10 and measuring the impedance between them. Based on a single time constant model, the interface between the mouth skin (inner side of the lips) and the fabric electrode may be represented by an equivalent circuit model. The skin-contact impedance has been measured three times for each of the eight subjects.

The EOG signal quality of the mouthguard 10 was verified on the eight participants by having them perform five different eye movements (blinking, looking to the left and right, and looking up and down). For each trial, the eye movement was repeated three times with a few seconds of rest (staring at the centre) in between. The participants were instructed to stay still and relax their face muscles during the experiment to minimize the amount of noise. All subjects have performed each eye activity in 5 trials. In each trial, the subjects have repeated the activity five times with resting (staring at a middle point) in between. Thus, each eye activity has been performed 25 times in total for each participant.

To demonstrate the long-term EOG data collection ability of the smart mouthguard, one of the subjects has worn the EOG mouthguard during sleep while the EOG data has been streamed to their nearby smartphone. No restriction in natural movements during sleep have been observed.

The skin-electrode impedance (magnitude and phase), contact parallel capacitance, and parallel resistance for the eight subjects has been measured for three different trials. Averaged overall all trials of all subjects, the skin-electrode impedance of the fabric electrode has the magnitude of 6.7 kilo Ohms and the phase of −26.32 degrees. Skin-electrode impedance value is low enough for good EOG signal quality. The averaged skin-electrode contact parallel capacitance is 0.26 micro F, and the averaged parallel resistance is about 7.59 kilo Ohms.

It has been observed that the skin-electrode contact impedance changes between trials (or over time). This change is likely due to the change in the amount of saliva inside mouth over time. The saliva may work like a conductive gel and may help reduce the impedance. Hence, saliva may help EOG measurements. Also, subjects may apply pressure on the electrodes by sucking on the mouthguard or moving their lips. This may cause the impedance to vary, but not significantly enough to affect the EOG measurements with the maximum change of 5.5 kilo Ohms between trials on the same subject.

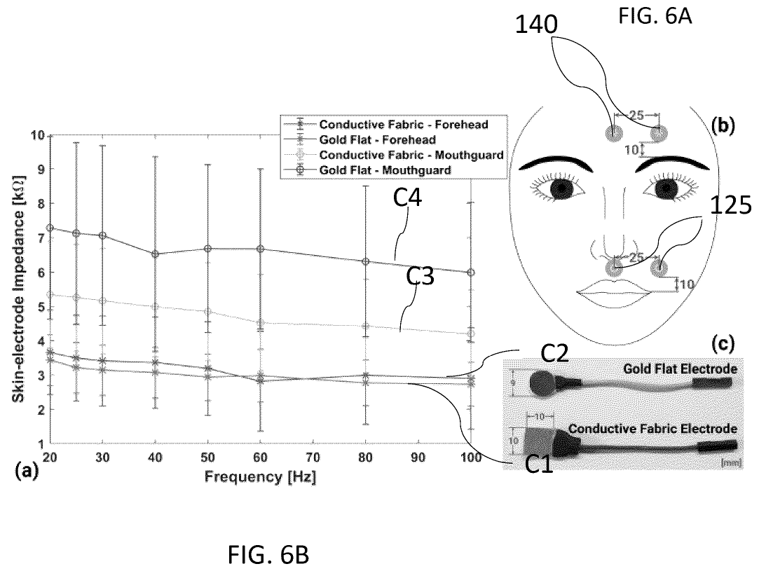

FIG. 6A illustrate a configuration used for testing. The configuration includes two face electrodes 140 and two mouth electrodes 125. The face electrodes 140 are mounted on a forehead of the user and may be standard gold flat electrodes. The two mouth electrodes 125 are disposed on the mouthguard 10. The electrodes 125, 140 may be standard gold flat electrodes or conductive fabric electrodes.

The impedance of the mouth electrodes 125 was compared to that of the face electrodes 140. Five measurements were done for each of the four electrode type-position combinations using the Keysight 4980A LCR meter at 5 mV over the frequency range of 20 Hz to 100 Hz. The impedance was measured between one electrode placed in the middle (of the forehead or the mouthguard) and another electrode placed 25 mm centre-to-centre to the left of it. For measurement on the forehead, the skin was first cleaned with NuPrep, and the electrodes were applied with Ten20 conductive paste. No gel or conductive paste was applied to the electrodes for measurements inside mouth as the saliva acted as the conductive medium.

FIG. 6B shows that the impedance of the two types of electrodes are very comparable on the forehead (see curves C1 and C2). Inside mouth the impedance of the fabric electrode on average was lower than that of the standard electrodes (see curves C3 and C4, respectively). This may be due to the fact that the fabric can absorb the saliva and curve along the mouthguard better. The average skin-electrode impedance of the flexible fabric electrodes was about 4.8 to 5.5 kΩ inside mouth, which met the standard for good EOG signal quality. Although the intra-oral EOG signals were weaker than the typical EOG signals measured on the forehead (around the eyes), the EOG patterns corresponding to different eye movements could be detected and distinguished from each other after processing the acquired intra-oral EOG signals. The EOG patterns due to horizontal eye movement (looking to the left or right) can be observed in the channels that measure the potential difference between the top electrodes and bottom electrodes as explained above with reference to FIG. 4A. Vertical-eye-movement EOG patterns (blinking and looking up or down) are recognized in the left or right electrode pairs. These signal patterns (see FIG. 4A) support the ocular dipole theory: the electrode that becomes closer to the eye cornea during the eye rotation has more positive potential.

### Board Evaluation

The skin-electrode impedance (magnitude and phase), contact parallel capacitance, and parallel resistance for the eight subjects has been measured for three different trials.

Frequency Response: The only component in the EOG board that amplifies the EOG signals is the amplifier 27. The PGA gains of all 4 channels were set to 24. The effective (or differential) gain of the board was evaluated by having two same-frequency sinusoid signals (from ac generator) of 180 degrees phase difference as inputs to the two electrodes of each channel. The frequency of the signals is varied from 0.1 to 100 Hz while keeping the amplitude at 90 mV. The BIAS electrode is connected to the ground of both sinusoid signals. The effective gain of the 3 EOG measuring boards is 23.18±4.22 (or 27.30±12.51 dB) in the EOG frequency band.

Common-Mode Rejection: The CMR response of a system, another metric, quantifies the ability of rejecting common-mode signals and interference by the system. Here, the EOG board is also tested for the CMR response. The common-mode rejection ratio (CMRR) of the EOG board is calculated as follows:

$$CMRR = 20 log ( \frac{{A}_{D}}{\vert {A}_{C} \vert} ) [ dB ]$$

in which A_{D} is the differential voltage gain which is already measured above. Ac is the common-mode voltage gain which is measured in the same way, except that the two measuring electrodes of each channel are connected together and to the same sinusoid signal. The CMRR of three characterized EOG measuring boards is about 103.17±4.60 dB in the frequency band of the EOG signals. Therefore, it is demonstrated that the EOG board has an excellent rejection response to common-mode input signals. The CMRR of the EOG measurement boards characterized by measurement considers the low-pass filter of each channel. Since the tolerances of the capacitor and resistor used for the low-pass filter are only ±1%, the tolerances of the capacitor and resistor will have very negligible effect on the CMRR of the EOG measurement board.

The real CMRR (or effective CMRR) during measurement is actually lower than the measured CMRR (103 dB) due to the input impedance mismatch in each EOG channel. This is because the electrodes are not perfectly identical and the skin-electrode impedance is susceptible to the change due to saliva amount and pressure applied on the electrodes. It is possible to consider only the resistive part of the electrodes' contact impedance for simplicity and ignore the reactive part of the electrodes' contact impedance. The voltage measured at each electrode is the combination of the differential signal VD and the common-mode signal V_{C}. V_{OUT} is the output voltage of the amplifier. According to the amplifier datasheet, the input impedance of the differential amplifiers in ADS1299 is 1 giga Ohms. It has been observed that the mismatch of input impedance of the electrodes decreases the effective CMRR to at least 93.6 dB, which is not too significant.

Noise Spectrum: The internal noise of the EOG board is measured by observing the output of a channel when connecting the two electrodes of each channel and the BIAS electrode together with no external inputs. The noise spectrum is obtained by applying fast-Fourier transform (FFT) to the time-domain output data of the channel. The noise spectrum of one of the 3 EOG boards demonstrates the low noise of the EOG boards. The noise magnitude of the 3 boards is 0.01±0.63 μV/v Hz over the EOG frequency range. Although the power line interference at 60 Hz and a small environment noise at 67 Hz (Channel 3) are visible in the noise spectrum, they are not in the EOG frequency band so can be filtered out easily. Besides that, most of the noise spectrum variance is due to the low-frequency components below 1 Hz.

Power Consumption and Charging: To measure the power consumption of the EOG board, a Keysight 34410A multimeter (in dc current mode) is connected in series with the positive supply of the LiPo battery to measure the current. Additionally, an NI USB-6009 (in differential voltage mode) is connected in parallel with the battery to measure the voltage. The voltage and current measurements are recorded at 1 Hz over 11 h. As in FIG. 16, the board consumes 0 mA when the power switch is turned off, 10 mA when the EOG board is turned on and the BLE is advertising, and 16 mA when it BLE connects and streams the data. With a fully charged 3.7 V 150 mAh LiPo battery, the 3 characterized EOG boards can work for 9.75±0.32 h during continuous usage. The power transfer efficiency (PTE) of the EOG board and the transmitter is measured at different distances between the Rx and Tx coils. Similar to when measuring the power consumption, the charging current (at the battery) is measured by the Keysight 34410A multimeter and the charging voltage is measured by the NI USB-6009. The Keysight E3648A provides the 5 V power supply to the transmitter board while measuring its current at the same time.

For each Rx-Tx coil distance, the charging current and voltage and the supply current are measured for 2 min. The LiPo battery is discharged to about 2.5 V before every charging measurement. It was observed that the highest average PTE of the 3 boards is 25.88% at 5 mm distance which is then used for measuring the charging time. The battery can also be charged relatively efficiently in the range of 4-9 mm Tx-Rx coil distance.

To measure the wireless charging time of the battery using the EOG board, the EOG board is placed on top of the transmitter board with its Rx coil aligned with the Tx coil of the transmitter. The voltage and current data are recorded at 1 Hz over 8 h using the same setup as in the power consumption measurement (ammeter in series with the battery and voltmeter in parallel with the battery). The charging is performed when the battery is almost empty (not enough for the board to work) to when the battery is full. It was observed that the wireless charging of the 150 mAh 3.7 V LiPo battery takes about 7 h to complete. The battery charging current starts at 100 mA (the full programmed charge current) and slowly drops over time. On the other hand, the battery voltage starts at about 2.5 V and then increases over charging time. The LED connected to the CHRG pin of the LTC4124 IC blinks slowly to indicate successful charging. When the battery voltage almost reaches the set charging voltage of 4.0 V (after 4.76 h of charging), the LTC4124 slowly decreases the charging current and starts a 3-h charge termination timer. When the charging current drops to 10 mA (10% of the full programmed charge current), the indicator LED stops blinking. There is still charging current, but it gradually becomes very small. After 3 h since the charge termination timer starts, the LTC4124 stops the charging completely (at about 7-h mark).

### Electrodes

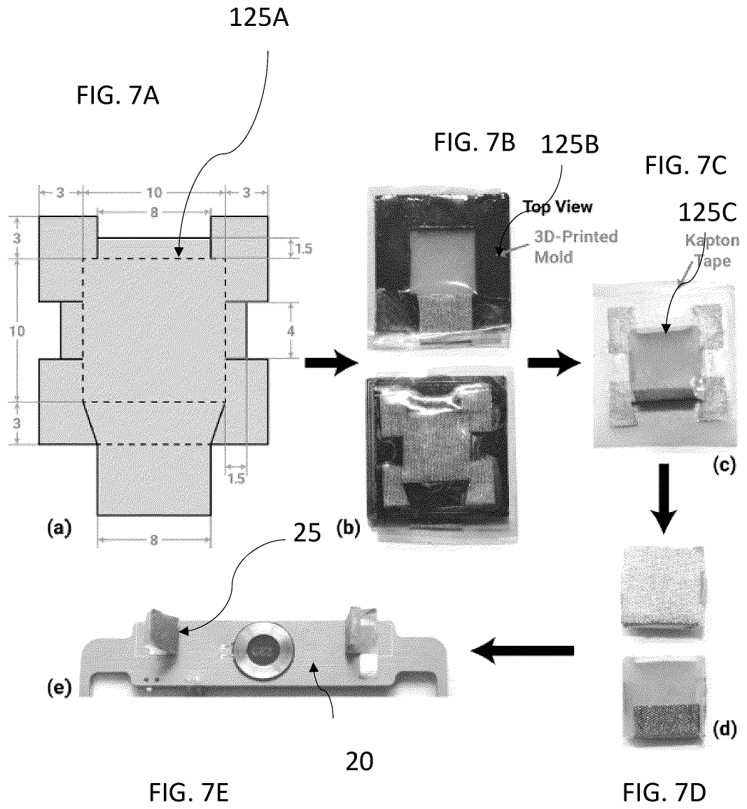

Referring to FIGS. 7A to 7D, a process for manufacturing the electrodes 25 is shown. The electrodes 25 may be made of the Adafruit woven silver-coating fabric (manufacturer product number of 1168). The conductive fabric 125A is first cut into the shape with dimensions shown in FIG. 7A, although other dimensions are contemplated. The cut fabric 125A is then folded and taped to a 3D-printed ABS mold 125B. Ecoflex 00-30 is poured into the mold 125B to form the soft base of the electrode 25. After the Ecoflex is cured, the electrode is removed from the mold 125B, and its edges are trimmed. Since the fabric cannot be soldered on directly, a fastener 125C, which may be a 5 mm×20 mm copper tape piece is first soldered to the electrode pad on the PCB before being taped to the electrode. In some embodiments, the top and bottom sub-mouthguards may be made of Realzeal™ EVA. They may be softened with hot water and then molded to a wearer's teeth.

### Single Jaw Mouthguard

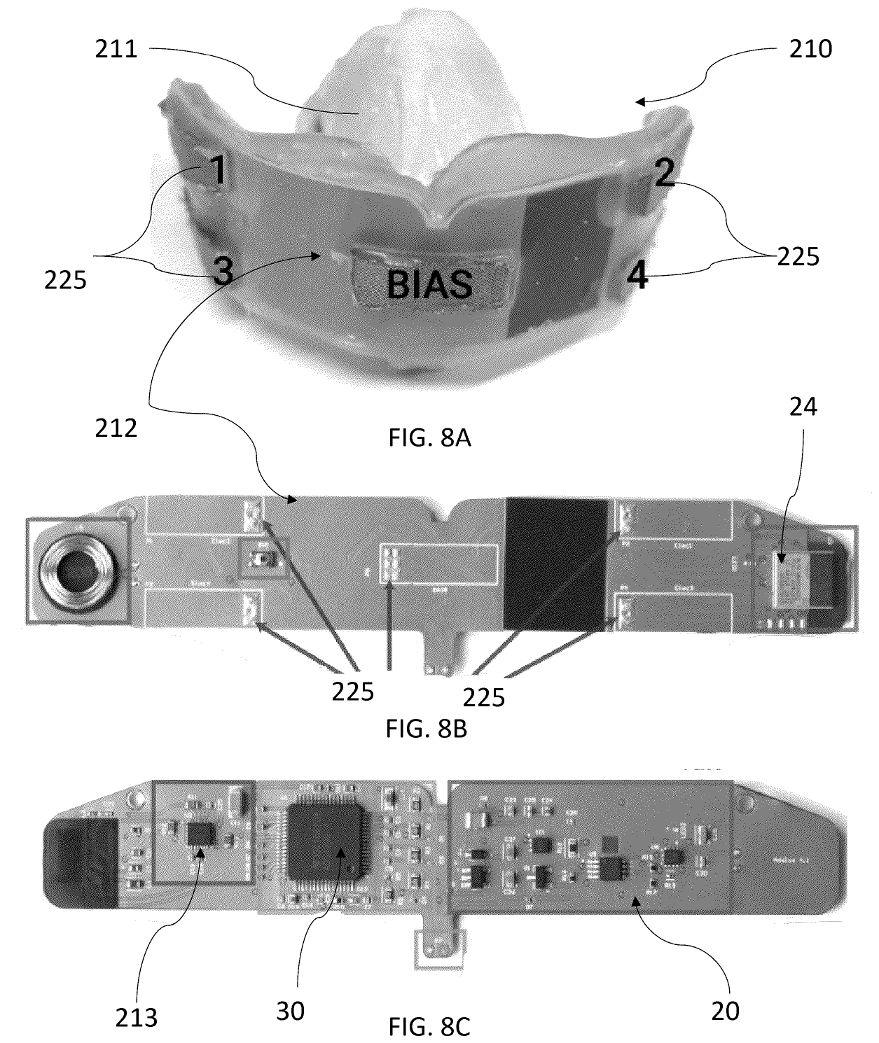

Referring to FIGS. 8A to 8C, another embodiment of a mouthguard is shown at 210. In this embodiment, the mouthguard 210 is configured to be worn either over the upper teeth or the lower teeth of the user. In this embodiment, the mouthguard 210 is designed to be worn over the upper teeth. Having only one “sub” mouthguard instead of two may be more comfortable to the user. The device 210 may include a body 211 made of ethylene-vinyl acetate (EVA) or any other suitable material. The device 210 includes five conductive fabric electrodes 225 (labelled 1 to 4 and BIAS in FIG. 8A). Device 210 includes a flexible measurement board 212.

The measurement board 212 has a front side shown in FIG. 8B and a back side shown in FIG. 8C. The electrodes 225 may be disposed on the front side while the measurement and charging circuit may be located on the back side. The device 210 may include an inertial measurement unit (IMU) sensor 213 affixed to the board 212 for artifact detection. Sensor 213 is operatively connected to the controller 30.

Due to the flexibility of the measurement board 212 and the electrodes 225, the board could be easily attached to the curved surface of the single jaw mouthguard. The intraoral EOG signals measured from the electrodes are low-pass filtered with passive RC filter at about 72 KHz. The filtered EOG signals are then passed through ADS1299 (a Delta-Sigma modulator followed by digital decimation filters) for amplification (at gain of 24), filtering, and digitization (at sampling rate of 250 Hz). Additionally, the ADS1299 performs common-mode rejection by driven-right-leg circuit. The digital signals are sent to the communication module.

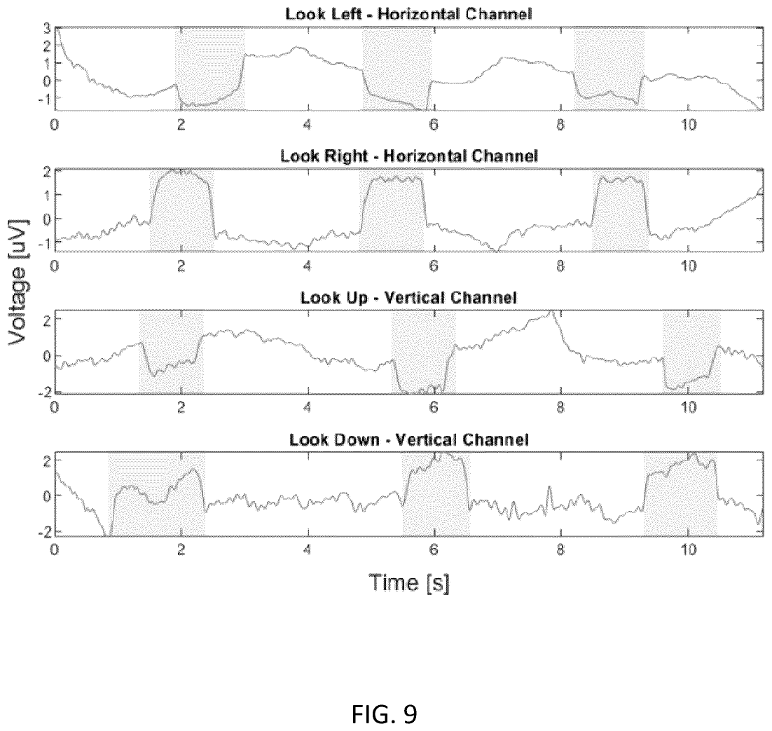

Referring to FIG. 9, the device 210 was tested on participants in which each participant performed 4 different eye movement activities (looking up, down, left, and right) with 15 times for each type. Multivariate Empirical Mode Decomposition (MEMD) was applied to the recorded intraoral EOG and IMU data to filter out the baseline drifts and high-frequency noises. MEMD is an extension of EMD that can decompose multichannel data into mode-aligned (same frequency) IMF components across channels. Only IMFs 5 to 9 were kept (which correspond to the frequency range of about 0.5 to 12 Hz) to reconstruct the filtered signals. As shown, different eye movement activities have unique MEMD-filtered intraoral EOG patterns which support the ocular dipole theory. During looking to the right, the cornea (with higher potential than the retina) becomes closer to electrode 2 than electrode 1, thus, the first horizontal channel will record an increase in the EOG signal. On the other hand, when looking up, the retina becomes closer to electrode 1 than electrode 3, which makes electrode 1 has more negative potential than electrode 3, thus, cause a decrease in the first vertical channel. Although the magnitude of the intraoral EOG signal is smaller than that when electrodes are placed around the eyes, all horizontal and vertical eye movement activities of the participants (15 examples for each activity) are distinguishable. Therefore, the eye activities for can be detected from the acquired EOG signal with an accuracy of 100%.

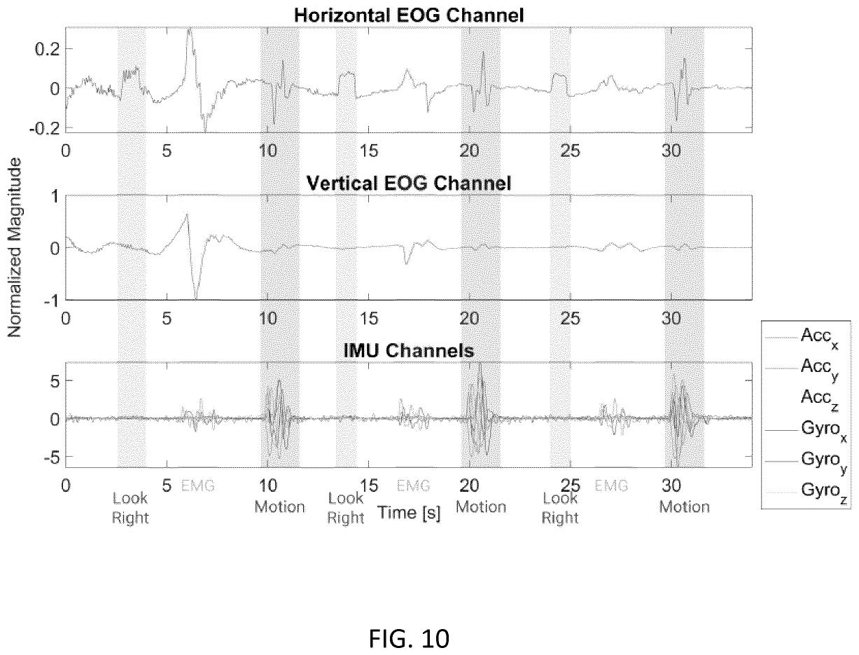

Referring to FIG. 10, EOG signals are susceptible to muscle (EMG) and motion artifacts as shown in the horizontal and vertical EOG channels. The IMU sensor 213 on the measurement board 212 can pick up the muscle movements and motion as shown in the IMU channel. The IMU data may be used to remove the EMG and motion artifacts from the EOG signals.

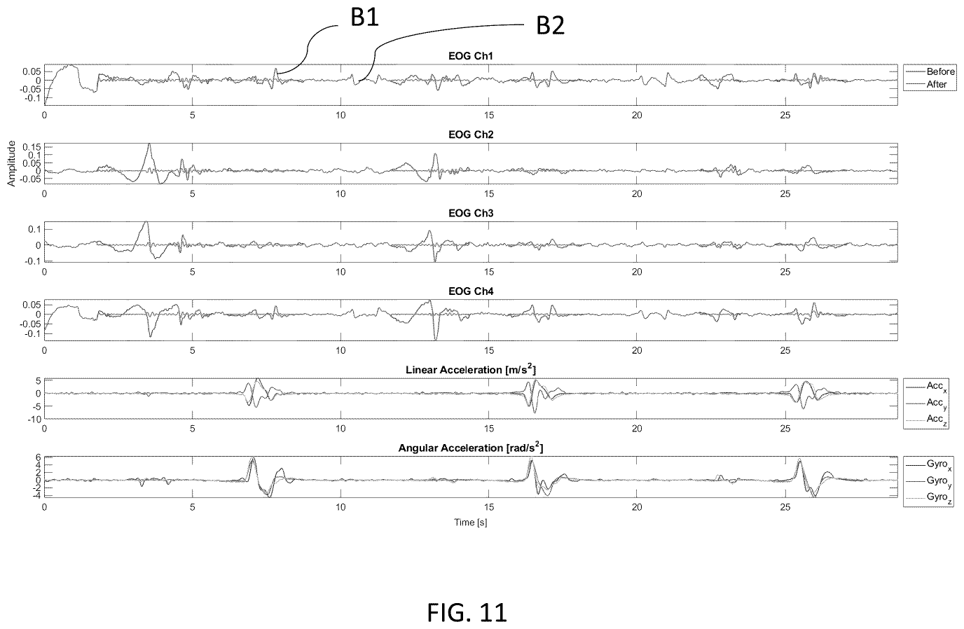

Referring to FIG. 11, the EGO patterns of each of the four electrodes 225 are shown for a user looking left. The two bottom graph illustrates linear acceleration and angular acceleration in each of the three directions (x, y, z). As shown, the user is clenching his/her teeth and rotating his/her head to the left. This causes artifacts. The signals obtained from the IMU sensor 213 may be used to correct the EOG patterns. The four first graphs illustrate each two curves B1, B2, namely, before and after the artifact removal. Using the IMU sensor 213 may thus allow to filter out movements of the user that are not correlated with eye movements caused by any of the sleep phases.

### Method

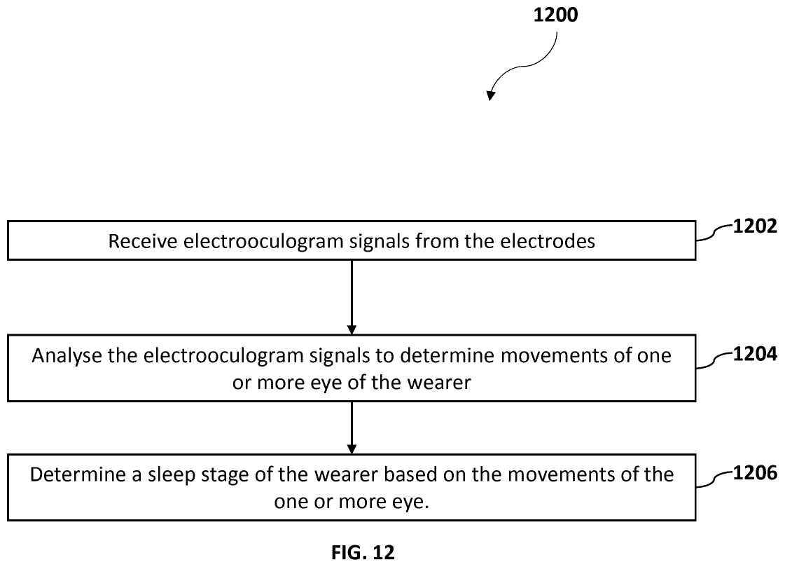

Referring now to FIG. 12, a method of analysing sleep using the mouthguard 10 of FIG. 2A or the mouthguard 210 of FIG. 8A is shown at 1200. The method 1200 may be performed by the controller 30. The method 1200 includes receive electrooculogram signals from the electrodes 25 at 1202; analyse the electrooculogram signals to determine movements of one or more eye 1 (FIG. 1A) of the wearer at 1204; and determine a sleep stage of the wearer based on the movements of the one or more eye 1 at 1206.

In the embodiment shown, the electrooculogram signals from the electrodes may be along four channels. The electrooculogram signals may be analysed by performing one or more of a wavelet decomposition (e.g., 4th order decomposition), a butterworth bandpass filtering, and a multivariate empirical mode decomposition, for each of the four channels. The bias electrode 25A may be used to remove noise from the signals. The method 1200 may thus include receiving a bias signal from the bias electrode 25A; and subtract the bias signal from the electrooculogram signals.

The method 1200 may determining that the sleep sage corresponds to: wake stage, stage 1 (N1), stage 2 (N2), stage 3 (N3), and Rapid Eye Movement (REM) stage.

In some embodiments, the analyse of the electrooculogram signals to determine the movements of the one or more eye includes: determine left and right movements of the one or more eye from potential differences between the EOG electrodes located on the top and/or between the EOG electrodes located on the bottom of the mouth guard; and determine up and down movements of the one or more eye from potential differences between the EOG electrodes located on the left side and/or between the EOG electrodes located on the right side of the mouth guard.

In some embodiments, the analyse of the electrooculogram signals includes: performing a fourth-order wavelet decomposition for each of four channels, each of the four channels associated with a respective one of the EOG electrodes.

The method 1200 may further include receive a bias signal from the bias electrode; and subtract the bias signal from the electrooculogram signals.

The method 1200 may further include: receive IMU signals from the IMU sensor 213; and remove artifacts from the electrooculogram signals using the IMU signals, the artifacts caused by one or more of jaw clenching, snoring, tongue movement, gulping, and head movements of the wearer.

### Controller

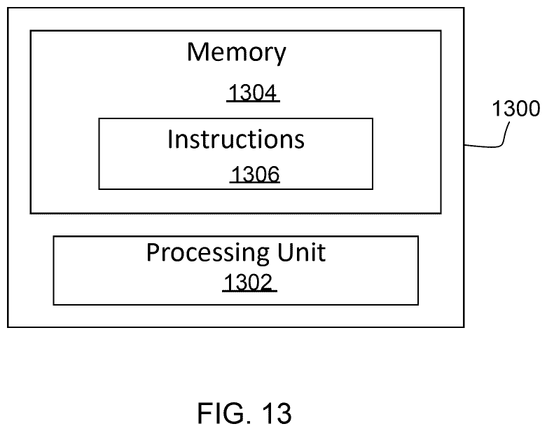

With reference to FIG. 13, an example of a computing device 1300 is illustrated. For simplicity only one computing device 1300 is shown but the system may include more computing devices 1300 operable to exchange data. The computing devices 1300 may be the same or different types of devices. The controller 30 may be implemented with one or more computing devices 1300.

The computing device 1300 comprises a processing unit 1302 and a memory 1304 which has stored therein computer-executable instructions 1306. The processing unit 1302 may comprise any suitable devices configured to implement the method described herein such that instructions 1306, when executed by the computing device 1300 or other programmable apparatus, may cause the functions/acts/steps performed as part of the method as described herein to be executed. The processing unit 1302 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 1304 may comprise any suitable known or other machine-readable storage medium. The memory 1304 may comprise non-transitory computer readable storage medium, for example, but not limited to, an electronic, magnetic, optical, electromagnetic, infrared, or semiconductor system, apparatus, or device, or any suitable combination of the foregoing. The memory 1304 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 1304 may comprise any storage means (e.g., devices) suitable for retrievably storing machine-readable instructions 1306 executable by processing unit 1302.

The methods and systems described herein may be implemented in a high-level procedural or object-oriented programming or scripting language, or a combination thereof, to communicate with or assist in the operation of a computer system, for example the computing device 1300. Alternatively, the methods and systems described herein may be implemented in assembly or machine language. The language may be a compiled or interpreted language. Program code for implementing the methods and systems described herein may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems described herein may also be considered to be implemented by way of a non-transitory computer-readable storage medium having a computer program stored thereon. The computer program may comprise computer-readable instructions which cause a computer, or more specifically the processing unit 1302 of the computing device 1300, to operate in a specific and predefined manner to perform the functions described herein, for example those described in the method described herein.

Computer-executable instructions may be in many forms, including program modules, executed by one or more computers or other devices. Generally, program modules include routines, programs, objects, components, data structures, etc., that perform particular tasks or implement particular abstract data types. Typically, the functionality of the program modules may be combined or distributed as desired in various embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

It is noted that various connections are set forth between elements in the preceding description and in the drawings. It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities. The term “connected” or “coupled to” may therefore include both direct coupling (in which two elements that are coupled to each other contact each other) and indirect coupling (in which at least one additional element is located between the two elements).

It is further noted that various method or process steps for embodiments of the present disclosure are described in the preceding description and drawings. The description may present the method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various aspects of the present disclosure have been disclosed, it will be apparent to those of ordinary skill in the art that many more embodiments and implementations are possible within the scope of the present disclosure. For example, the present disclosure as described herein includes several aspects and embodiments that include particular features. Although these particular features may be described individually, it is within the scope of the present disclosure that some or all of these features may be combined with any one of the aspects and remain within the scope of the present disclosure. References to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. The use of the indefinite article “a” as used herein with reference to a particular element is intended to encompass “one or more” such elements, and similarly the use of the definite article “the” in reference to a particular element is not intended to exclude the possibility that multiple of such elements may be present.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

1. A mouthguard for measuring eye movements of a wearer, comprising:
a body adapted to engage teeth of the wearer, the body extending laterally along a frontal axis between a left side and a right side and extending vertically along a vertical axis normal to the frontal axis, between a top and a bottom;
a power source;
electrooculogram (EOG) electrodes secured to the body and operatively connected to the power source, the EOG electrodes including a top right electrode on the right side and adjacent the top of the body, a top left electrode on the left side and adjacent the top, at least one bottom electrode mounted adjacent the bottom of the body, and a bias electrode being substantially centered on the body between the left side and the right side; and
a controller operatively connected to the EOG electrodes and to the power source, the controller having a processing unit and a computer-readable medium operatively connected to the processing unit and having instructions stored thereon executable by the processing unit to:
receive electrooculogram signals from the EOG electrodes; and
analyse the electrooculogram signals to determine movements of one or more eye of the wearer.

2. The mouthguard of claim 1, wherein the at least one bottom electrode includes: a bottom right electrode on the right side and adjacent the bottom of the body; and a bottom left electrode on the left side and adjacent the bottom of the body.

3. The mouthguard of claim 1, wherein the computer-readable medium has instructions executable by the processing unit to determine a sleep stage of the wearer based on the movements of the one or more eye determined by the electrooculogram signals.

4. The mouthguard of claim 3, wherein the computer-readable medium has instructions executable by the processing unit to determine the sleep stage by determining that the sleep stage corresponds to: wake stage, stage 1 (N1), stage 2 (N2), stage 3 (N3), and Rapid Eye Movement (REM) stage.

5. The mouthguard of claim 1, wherein the EOG electrodes include conductive fabric electrodes.

6. The mouthguard of claim 1, wherein the computer-readable medium has instructions executable by the processing unit to receive the electrooculogram signals from the EOG electrodes via four channels.

7. The mouthguard of claim 6, wherein the computer-readable medium has instructions executable by the processing unit to analyse the electrooculogram signals by performing a fourth-order wavelet decomposition for each of the four channels.

8. The mouthguard of claim 1, wherein the computer-readable medium further instructions executable by the processing unit to:
receive a bias signal from the bias electrode; and
subtract the bias signal from the electrooculogram signals.

9. The mouthguard of claim 1, comprising a wireless communication module for data transmission.

10. The mouthguard of claim 1, further comprising an inertial measurement unit (IMU) sensor mounted to the body, the IMU sensor operatively connected to the controller, the computer-readable medium further having instructions executable by the processing unit to:
receive IMU signals from the IMU sensor; and
remove artifacts from the electrooculogram signals using the IMU signals, the artifacts caused by one or more of jaw clenching, snoring, tongue movement, gulping, and head movements of the wearer.

11. The mouthguard of claim 1, wherein the power source is a battery mounted to the body.

12. The mouthguard of claim 11, comprising an inductive charging coil mounted to the body and operatively connected to the battery, the inductive charging coil configured to wirelessly recharge the battery.

13. The mouthguard of claim 1, wherein the body is configured to be engaged solely by either of upper teeth and lower teeth of the wearer.

14. The mouthguard of claim 1, wherein the body has an upper part and a lower part, the upper part configured to engage upper teeth of the wearer, the lower part configured to engage lower teeth of the wearer.

15. A method for analysing sleep, the method comprising:
receiving electrooculogram signals from EOG electrodes within a mouthguard worn by a wearer;
analysing the electrooculogram signals to determine movements of one or more eye of the wearer; and
determining a sleep stage of the wearer based on the movements of the one or more eye.

16. The method of claim 15, wherein the mouthguard has a left side, a right side, a top, and a bottom, the EOG electrodes including a top right electrode on the top and right side, a top left electrode on the top and left side, a bottom right electrode on the bottom and right side, and a bottom left electrode on the bottom and left side, said analysing of the electrooculogram signals to determine the movements of the one or more eye includes:
determine left and right movements of the one or more eye from potential differences between the EOG electrodes located on the top and/or between the EOG electrodes located on the bottom of the mouthguard; and
determine up and down movements of the one or more eye from potential differences between the EOG electrodes located on the left side and/or between the EOG electrodes located on the right side of the mouthguard.

17. The method of claim 15, wherein the determining of the sleep stage of the wearer based on the movements of the one or more eyes includes determine that the sleep stage corresponds to one or more of: wake stage, stage 1 (N1), stage 2 (N2), stage 3 (N3), and Rapid Eye Movement (REM) stage.

18. The method of claim 15, wherein the analysing of the electrooculogram signals includes:
performing one or more of a wavelet decomposition, a butterworth bandpass filtering, and a multivariate empirical mode decomposition, for each of four channels, each of the four channels associated with a respective one of the EOG electrodes.

19. The method of claim 15, wherein the EOG electrodes further include a bias electrode, the method comprising:
receive a bias signal from the bias electrode; and
subtract the bias signal from the electrooculogram signals.

20. The method of claim 15, wherein the mouthguard further includes an inertial measurement unit (IMU) sensor, the method comprising:
receive IMU signals from the IMU sensor; and
remove artifacts from the electrooculogram signals using the IMU signals, the artifacts caused by one or more of jaw clenching, snoring, tongue movement, gulping, and head movements of the wearer.
